(19) United States
(12) Patent Application Publication
(10) Pub. No.: US 2024/0177812 A1
(43) Pub. Date: May 30, 2024

(54) SYSTEMS AND METHODS FOR EXPEDIENT COMPUTATION OF SUBSTRATE DEFORMATIONS IN THERMO-COMPRESSION BONDING

(71) Applicants: Marc Mignolet, Phoenix, AZ (US); Xiaoquan Wang, Chandler, AZ (US)

(72) Inventors: Marc Mignolet, Phoenix, AZ (US); Xiaoquan Wang, Chandler, AZ (US)

(73) Assignee: Arizona Board of Regents on Behalf of Arizona State University, Tempe, AZ (US)

(21) Appl. No.: 18/525,699

(22) Filed: Nov. 30, 2023

Related U.S. Application Data
(60) Provisional application No. 63/428,874, filed on Nov. 30, 2022.

(51) Int. Cl. G16C 60/00; G16C 20/70; G16C 20/80

(52) U.S. Cl. CPC G16C 60/00; G16C 20/70; G16C 20/80

(57) ABSTRACT

A system can apply a Global Reduced Order Modeling (ROM), a Zonal ROM, or a Layer-By-Layer analysis method to accelerate computations of a static displacement field of a substrate during modeling of thermo-compression bonding. The system fully takes into account a glass transition of dielectric substrates as well as nonhomogeneity of the various layers.

### CROSS-REFERENCE TO RELATED APPLICATIONS

This is a U.S. Non-Provisional Patent Application that claims benefit to U.S. Provisional Patent Application Ser. No. 63/428,874 filed 30 Nov. 2022, which is herein incorporated by reference in its entirety.

### FIELD

The present disclosure generally relates to thermocompression of solid parts, e.g., a die, on substrates, and in particular, to a system and associated method for expedient computations of substrate deformations in thermo-compression bonding.

### BACKGROUND

Mechanical analysis of thermo-compression bonding requires a complex modeling including a large volume of finite element computations. Each of these finite element computations (usually implemented using finite element analysis software such as Abaqus) is very time consuming, on the order of hour, which thus may limit the number of snapshots of the temperature considered but also prevents a rapid turn-around of predictions when redesigning the substrate structural layout.

It is with these observations in mind, among others, that various aspects of the present disclosure were conceived and developed.

### BRIEF DESCRIPTION OF THE DRAWINGS

FIG. 1A is an illustration showing application of pressure and

heat to an electronic substrate;

FIG. 1B is an illustration showing deformation of the electronic substrate of FIG. 1A resulting from application of heat;

FIG. 2A is a block diagram showing a Global Reduced Order Modeling methodology for predicting heat-induced deformation (spatial displacement) of a substrate having an ensemble of zones (e.g., layers) represented by an ensemble finite element model;

FIG. 2B is a simplified diagram showing construction of a set of stiffness coefficients of a predictive model for predicting heat-induced deformation;

FIGS. 3A-3C are a series of process flow diagrams showing a first process for predicting heat-induced deformation (spatial displacement) of a substrate according to the Global Reduced Order Modeling methodology of FIG. 2A;

FIG. 4 is a block diagram showing a Zonal Reduced Order Modeling methodology for predicting heat-induced deformation (spatial displacement) of a substrate having an ensemble of zones (e.g., layers) represented by an ensemble finite element model;

FIGS. 5A-5C are a series of process flow diagrams showing a second process for predicting heat-induced deformation (spatial displacement) of a substrate according to the Zonal Reduced Order Modeling methodology of FIG. 4;

FIG. 6A is an illustration showing a modeled substrate having a plurality of layers;

FIG. 6B is an illustration showing layer-by-layer deformation prediction of the modeled substrate of FIG. 6A resulting from application of heat for application of a Layer-by-Layer modeling methodology;

FIGS. 7A and 7B are a pair of process flow diagrams showing a third process for predicting heat-induced deformation (spatial displacement) of a substrate according to the Layer-by-Layer modeling methodology outlined with respect to FIGS. 6A and 6B; and

FIG. 8 is a simplified diagram showing an example computing system for implementation of the processes of FIGS. 3A-3C, 5A-5C, and/or 7A and 7B.

Corresponding reference characters indicate corresponding elements among the view of the drawings. The headings used in the figures do not limit the scope of the claims.

### DETAILED DESCRIPTION

The present disclosure is directed to systems and method for expedient computation of static displacements of a substrate subjected to a series of temperature distributions. In particular, the present disclosure outlines systems and methods for determining deformations taking place due to heating of a substrate, e.g., during thermal compression bonding, and more specifically on the development of strategies that reduce the computational effort and/or enable an increase parallelization of these computations. Methods outlined herein include three options to carry out the analysis which are based on: (i) a global structural reduced order model (ROM) in linear thermoelasticity, (ii) a zonal reduced order models of each layer and their assembly; and (iii) a computational strategy determining the deformations layer-by-layer using finite element analysis. Each option has its own value in different circumstances depending on the needs of the specific practical application of these methods, especially depending on available data and complexity of the substrate.

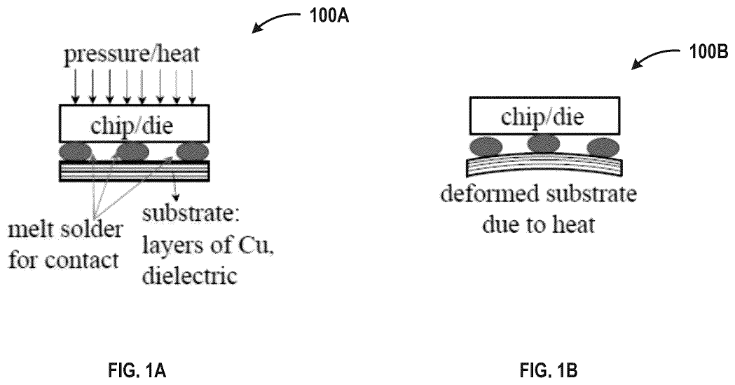

The lack of a rapid structural prediction has prevented the appropriate coupling between the substrate structural displacements and the heating from the die, i.e., the capture of the time and temperature varying contact between the die and the substrate top layer. Deformation of a multi-layered substrate is illustrated in FIGS. 1A and 1B, where FIG. 1A shows a pre-deformation modeled substrate 100A and FIG. 1B shows a post-deformation modeled substrate 100B. As the top layer deforms due to temperature, this contact changes because of the slight waviness of this layer and accordingly, the heat conduction is also modified until a full contact takes place.

### 1. Introduction

Mechanical analysis of a thermo-compression bonding of a die on a substrate starts with a transient thermal analysis to determine a temperature through the substrate due to the heating. A series of time snapshots of this evolving temperature field are then selected and applied to a structural finite element model to determine the corresponding displacement fields. This model is very complex involving up to multiple millions of degrees of freedom to reflect: (i) the layered structure of the substrate; and (ii) the nonhomogeneity of each layer (varying composition of copper and dielectric) but to also to capture the local structural properties which are strongly dependent on the local temperature, especially considering the glass transition of the dielectric.

Several key observations can be made. Specifically, regarding temperature, it was observed that the distribution of temperature of each of the layers is fairly constant as a function of time, especially once a full contact is established, but the magnitude of these distributions is the most significant difference between one time snapshot and another. A principal component analysis (PCA) has indeed revealed that one eigenvector is very largely dominant during the entire process with two more eigenvectors participating much less. During the beginning of the bonding process when a full contact is not yet achieved, one additional eigenvector will possibly be necessary to capture the nonuniformity of the temperature due to the partial contact.

A somewhat similar situation is also observed for the displacement fields obtained for different substrates and different dies, i.e., they are typically very similar in form but vary in magnitude. Moreover, it can be observed that the key displacement of interest is the one from the substrate top layer.

### 2. Reduced-Order Modeling Overview

To obtain the most compact modeling of the substrate deformations during the bonding process, it is important to also characterize the temperature distribution (the driver of the displacements) in its simplest form. The observations of the previous sections demonstrate first that the temperature distribution for a particular substrate can be expressed as:

T(x,y,z,t)=τ₁(t)Ψ₁(x,y,z)+τ₂(t)Ψ₂(x,y,z)+ (1)

where T(x, y, z, t) denotes the temperature at a spatial location (x, y, z) (z being through thickness) of the substrate at time t and Ψᵢ(x, y, z) are the temperature distributions in each layer obtained from PCA (in this context, synonymous with Proper Orthogonal Decomposition (POD)), i.e., eigenvectors i=1, 2, . . . . From the observations discussed above, it is expected that there is between 1 and 4 terms needed in Eq. (1) depending on accuracy. Moreover, the variables τᵢ(t), are often referred to as “temperature generalized coordinates” but also as temperature factors in the sequel capture the time evolution of the temperature.

The observations made with respect to the structural deformations and the representation of the temperature in the form of Eq. (1) suggest that the displacement field u(x, y, z, t) of the finite element mesh may be expressed as:

u(x,y,z,t)=q₁(t)U₁(x,y,z)+q₂(t)U₂(x,y,z)+ (2)

In Eq. (2), t is the time corresponding to the snapshot of temperature applied and the variables qᵢ(t) are often referred to as the “structural generalized coordinates” but also as displacement factors in the sequel which quantify the overall level of deformations. The “structural basis functions” Uᵢ capture the spatial variations of the deformations, they are akin to the PCA eigenvectors Ψᵢ(x, y, z).

Equations (1)-(2) form a “reduced order model” of the temperature and displacement in that the millions of degrees of freedom of the original finite element models (structural and thermal) have been reduced to a much smaller number of factors, i.e., the variables τᵢ(t) and qᵢ(t).

The reduced order modeling process is completed by inserting Eqs (1)-(2) in linear thermoelasticity, as only small displacements are observed and thus nonlinear geometric effects can be neglected. This effort leads to linear equations of the form:

k₁₁(τ)q₁+k₁₂(τ)q₂+ . . . =F₁(τ)

k₂₁(τ)q₁+k₂₂(τ)q₂+ . . . =F₂(τ) (3)

where kᵢⱼ(τ) are the stiffnesses of the various structural modes and are dependent on the temperature factors τᵢ(t) (lumped in the vector τ) since the structural properties depend on temperature, especially in view of the glass transition. Finally, the terms Fᵢ(τ) represent the thermal loading which also depend on the thermal generalized coordinates.

The great efficiency of ROMs results from the very small number of equations (3) in comparison to those of the full finite element model. However, this conclusion is based on the assumption that (i) the basis functions (structural) involved Uᵢ_{ⁱ}; (ii) the stiffnesses kᵢⱼ(τ); and (iii) the thermal loading coefficients Fᵢⱼ can all be determined very efficiently.

The present effort centers on:
the efficient determination of the structural basis functions Uᵢ_{ⁱ}, stiffnesses kᵢⱼ(τ), and thermal loading coefficients Fᵢⱼ from an assumed set of training data including displacements and temperatures distributions; and
the application of the ROM methodology and its validation.

The systems outlined herein can be applied under a “Global ROM” methodology, a “Zonal ROM Methodology”. An additional method that is not based on reduced order modeling, referred to as the “Layer-By-Layer” finite element modeling methodology will also be described. The results presented for the Global ROM demonstrate that structural reduced order models that include the temperature effects are indeed applicable and will provide excellent predictions of the displacement fields in the substrate. However, the cost of constructing such ROMs, i.e., determining the basis and identifying the coefficients of the model may require a computational effort similar to carrying out a limited number, e.g., 10, of full static solutions. Thus, this Global ROM approach would be applicable if a very detailed evolution of the displacements as a function of the changing temperature distribution is desired.

The layer-by-layer finite element modeling methodology determines the displacements by groups of 2-layers starting at the bottom toward the top. It effectively views the substrate as a weakly coupled chain of substructures (each being 2 consecutive layers), the weak coupling resulting from the low mechanical properties of an alternate layer. This approach was shown to lead to excellent predictions of the displacements, especially of the top layers, at a computational time that is only 32% of the one necessary for the full simulations. Moreover, since the finite element models involved are much smaller, the necessary memory is also dramatically reduced to 4% to 7% of the one necessary for the full solutions. This low memory need allows for the parallel computation of the response of the substrate to all temperature loadings at the same time vs. one at a time. Proceeding in this manner then reduces the wall time needed to get all solution to only 3% of the time with the full solution.

The capability to accurately determine the response in the layer-by-layer format suggests that Zonal ROMs, i.e., reduced order models of small parts of the substrate, e.g., 1 or 2 layers, can be conceived and potentially assembled to produce extremely fast predictions of the response. The present disclosure demonstrates that a unique basis can be found that represents the displacements of all of the layers (regardless of material) for all temperature cases (regardless of top or bottom of the substrate and of the temperature applied). This is a very important finding that suggests that this same basis could also be used for other chips thereby eliminating the need to run full finite element analysis solutions to construct the basis as in the Global ROM approach. The identification of the coefficients of these Zonal ROMs are also facilitated by the small size of the associated finite element model. Zonal ROM considers each layer independently and thus requires an appropriate handling of the necessary continuity of the displacements across layers.

A model substrate considered herein for validation has a basic structure with alternating layers from the free top (layer 1) to the bottom (layer 21) where the z displacements are imposed to be zero and the in-plane displacements are restricted at three points to block rigid body motions.

### 3. Global Reduced Order Modeling

A processor implementing Global ROM can receive, at the processor, a set of model parameters expressive of a modeled substrate having a plurality of layers, a set of simulation parameters and a set of physical properties. The processor can then determine a basis indicative of displacement for the ensemble of layers of the modeled substrate for a plurality of temperature cases through a Proper Orthogonal Decomposition (POD) of ensemble of displacements of all layers obtained in existing finite element analysis solutions. The processor can determine a set of displacement factors for the ensemble of layers of the modeled substrate using the set of model parameters and the set of simulation parameters through application of a global structural reduced order modeling methodology.

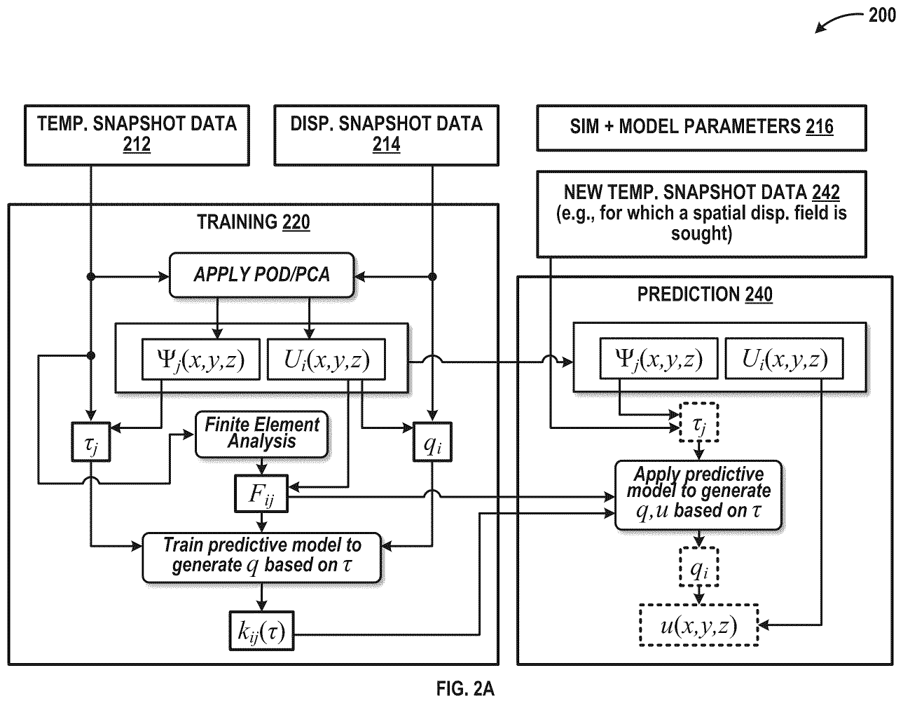

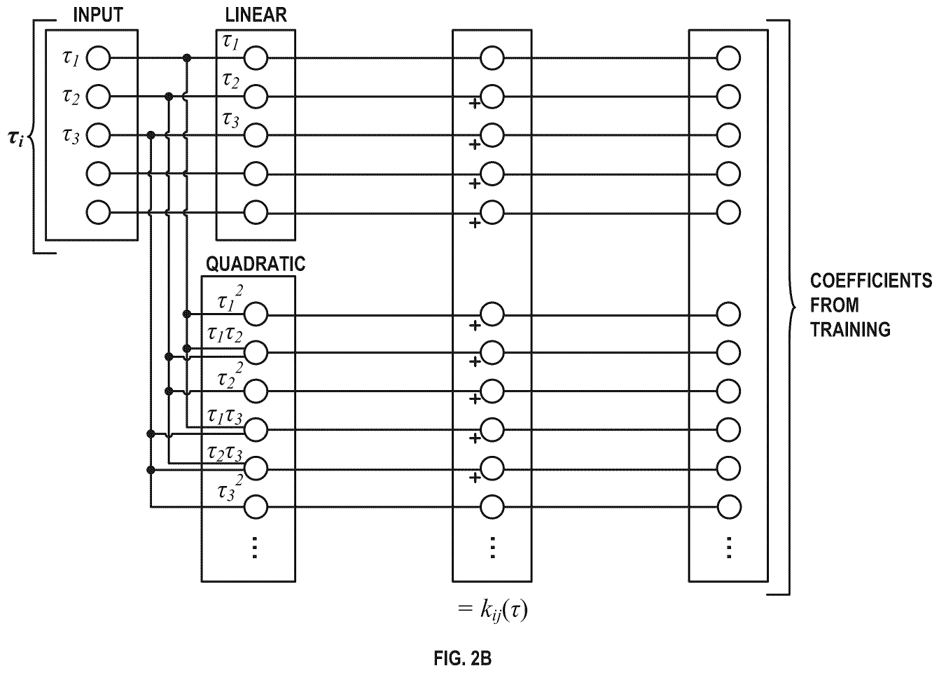

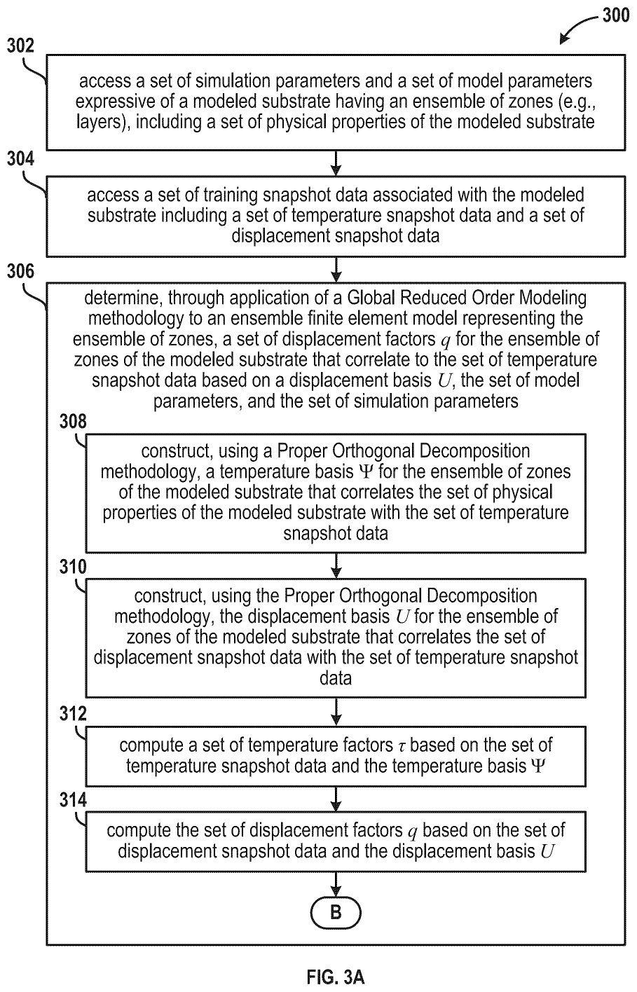

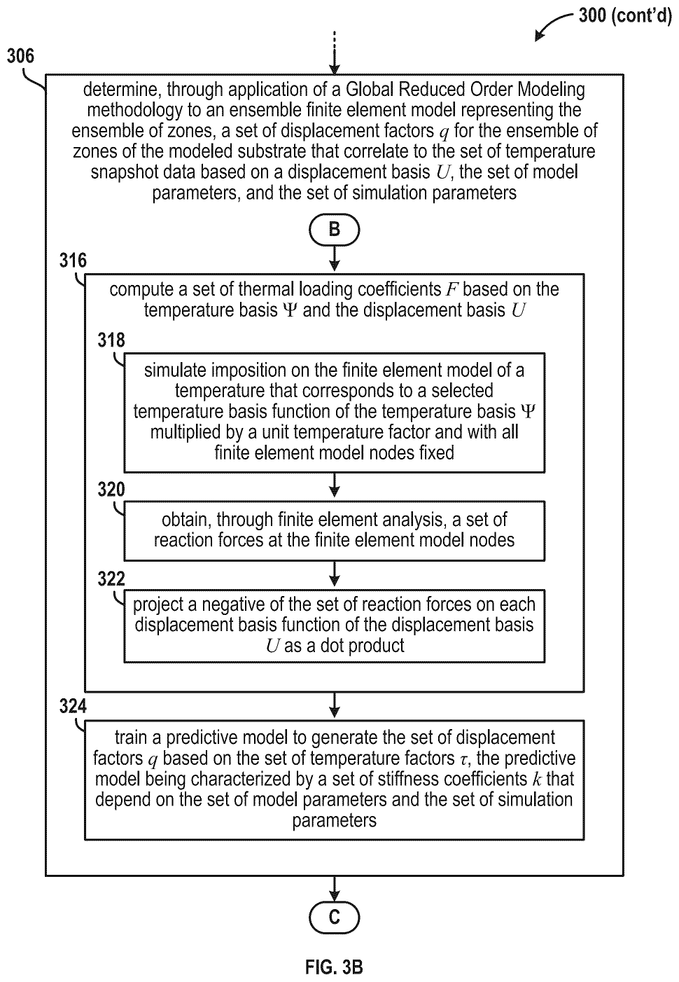

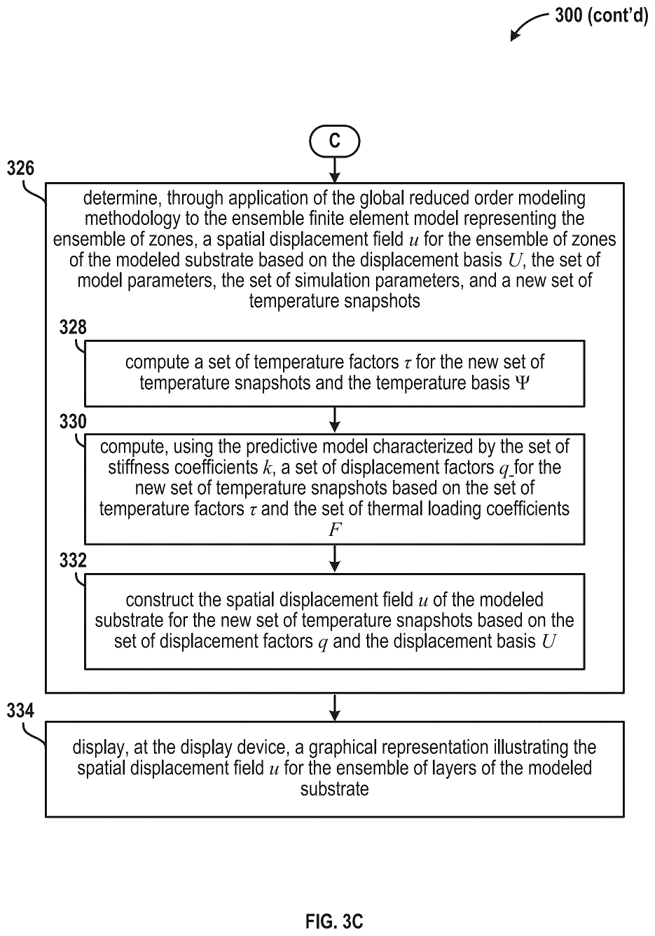

To determine a spatial displacement field u(x, y, z) for a modeled substrate having an ensemble of zones (or more specifically, layers), a computing device implementing Global ROM 200 conceptually illustrated in FIG. 2A and outlined as a first process of FIGS. 3A-3C can first access a set of simulation parameters and a set of model parameters expressive of the modeled substrate, including a set of physical properties of the modeled substrate. For Global ROM, the whole modeled substrate is represented in one shot for both temperature and displacement. The Global ROM system can include a “training” block 220 and a “prediction” block 240 as shown in FIG. 2A. Also, in FIG. 2A, boxes that are in dotted lines (the set of temperature factors τⱼ), the set of displacement factors qᵢ, and the spatial displacement field u(x, y, z) within the “prediction” block 240) are quantities that can change when new data is being applied. In contrast, the thermal loading coefficients Fᵢⱼ and the set of stiffness coefficients kᵢⱼ can remain constant once training is performed within the “training” block 220.

Training data employed by the “training” block 220 can include “snapshots” of temperature distribution (“a set of temperature snapshot data 212”) throughout a modeled substrate, as well as “snapshots” of structural deformation (“a set of displacement snapshot data 214”) throughout the modeled substrate that correlate with temperature distribution. The set of displacement snapshot data 214 and the set of temperature snapshot data 212 for the modeled substrate can be obtained through existing finite element analysis solutions of the modeled substrate. Other information used by the “training” block 220 can include simulation parameters and model parameters (shown in FIG. 2A as “Sim+Model Parameters 216). Based on the set of temperature snapshot data 212, the set of displacement snapshot data 214, the computing device can construct, using a Proper Orthogonal Decomposition methodology, a temperature basis Ψ and a displacement basis U for the ensemble of zones of the modeled substrate that correlates the set of displacement snapshot data 214 with the set of temperature snapshot data 212. By applying finite element analysis to the substrate in view of the temperature basis Ψ and the displacement basis U, the computing device can find a set of temperature factors τ and a set of displacement factors q for the modeled substrate that correspond with one another. The set of displacement factors q can then be used to train a predictive model to generate sets of displacement factors q based on a given set of temperature factors τ, illustrated in FIG. 2B. The set of displacement factors q are dependent upon the set of temperature factors τ expressive of a distribution, the set of model parameters, and the set of simulation parameters. Further, the set of displacement factors q can be determined from a set of stiffness coefficients (of a stiffness matrix k) and a set of thermal loading coefficients F.

Once the predictive model is trained, at the “prediction” block 240, the temperature basis Ψ and the displacement basis U can be used to generate a new set of displacement factors q in view of more temperature cases, e.g., in view of a new set of temperature snapshot data 242. The new set of displacement factors q can be used along with the displacement basis U to generate the spatial displacement field u(x, y, z) for a modeled substrate.

FIGS. 3A-3C show a first process 300 that includes steps 302-324 performed with respect to the “training” block 220 and steps 326-332 performed with respect to the “prediction” block 240 of FIG. 2A.

Referring to FIG. 3A, step 302 of first process 300 includes accessing a set of simulation parameters and a set of model parameters expressive of a modeled substrate having an ensemble of zones (or, more specifically, layers), including a set of physical properties of the modeled substrate. Step 304 includes accessing a set of training snapshot data associated with the modeled substrate including a set of temperature snapshot data and a set of displacement snapshot data. Step 306 includes determining, through application of a Global Reduced Order Modeling methodology to an ensemble finite element model representing the ensemble of zones, a set of displacement factors q for the ensemble of zones of the modeled substrate that correlate to the set of temperature snapshot data based on a displacement basis U, the set of model parameters, and the set of simulation parameters.

Step 306 can include several sub-steps 308-324. Steps 308 and 310 pertain to the basis functions for temperature and displacement that can be applied across the entire substrate. Step 308 includes constructing, using a Proper Orthogonal Decomposition methodology, a temperature basis Ψ for the ensemble of zones of the modeled substrate that correlates the set of physical properties of the modeled substrate with the set of temperature snapshot data. Step 310 includes constructing, using the Proper Orthogonal Decomposition methodology, the displacement basis U for the ensemble of zones of the modeled substrate that correlates the set of displacement snapshot data with the set of temperature snapshot data. In some embodiments, the displacement basis U is a split basis in which one or more modes of a plurality of modes of the displacement basis U include a set of in-plane components along an x-direction and a y-direction of the modeled substrate, and one or more modes of the plurality of modes of the displacement basis U include a set of transverse components along a z-direction of the modeled substrate.

Steps 312, 314 and 316 pertain to determination of temperature factors τ that correspond to the temperature basis Ψ, displacement factors q that correspond to the displacement basis U, and a set of thermal loading coefficients F that correlate the temperature basis with the displacement basis U. Step 312 includes computing a set of temperature factors τ based on the set of temperature snapshot data and the temperature basis Ψ (using projection, e.g., a dot product or a least-squares process). Step 314 includes computing a set of displacement factors q based on the set of displacement snapshot data and the displacement basis U (using projection, e.g., a dot product or a least-squares process). In FIG. 3A, step 314 concludes at (B).

Starting at (B) in FIG. 3B, step 316 includes computing a set of thermal loading coefficients F based on the temperature basis Ψ and the displacement basis U. Step 316 can include several sub-steps 318-322. Step 318 includes simulating imposition on the finite element model of a temperature that corresponds to a selected temperature basis function of the temperature basis Ψ multiplied by a unit temperature factor and with all finite element model nodes fixed. Step 320 includes obtaining, through finite element analysis, a set of reaction forces at the finite element model nodes. Step 322 includes projecting a negative of the set of reaction forces on each displacement basis function of the displacement basis U as a dot product. Step 316 results in one set of training thermal loading coefficients F for each pair of temperature basis function and displacement basis function (e.g., an ij-th set of training thermal loading coefficients Fᵢⱼ for a j-th temperature basis function Ψ paired with an i-th displacement basis function Uᵢ).

Following step 316 (including sub-steps 318-322), step 324 includes training a predictive model to generate the set of displacement factors q based on the set of temperature factors τ. With additional reference to FIG. 2B, the predictive model can be characterized at least in part by a set of stiffness coefficients k that depend on the set of model parameters and the set of simulation parameters. Each displacement factor qᵢ can be written as linear combination of the training thermal loading coefficients Fᵢⱼ corresponding to that displacement basis function Uᵢ, scaled by the corresponding set of temperature factors τⱼ. The coefficients of the linear combination form the inverse of a stiffness matrix k, the elements of which are polynomials of the set of temperature factors τ. The act of training the predictive model determines the coefficients of the polynomials in the temperature factors τ of the elements of the stiffness matrix k that lead to the smallest error between the displacement factors q obtained by the predictive model and those found in step 314. FIG. 3B concludes at (C), and the first process 300 continues at (C) of FIG. 3C.

Referring to FIG. 3C, the basis functions and the trained predictive model can then be applied to a new set of temperature snapshots (e.g., new set of temperature snapshot data 242) for construction of a spatial displacement field u of the modeled substrate for the new set of temperature snapshot data. Steps 326-332 of first process 300 pertain to the “prediction” block 240 of FIG. 2A.

Step 326 includes determining, through application of the global reduced order modeling methodology to the ensemble finite element model representing the ensemble of zones, a spatial displacement field u for the ensemble of zones of the modeled substrate based on the displacement basis U, the set of model parameters, the set of simulation parameters, and a new set of temperature snapshots. Step 326 can include several sub-steps 328-332. Step 328 can include computing a set of temperature factors τ for the new set of temperature snapshots and the temperature basis function Ψ (using projection, e.g., a dot product or a least-squares process). Step 330 can include computing, using the predictive model characterized by the set of stiffness coefficients k, a set of displacement factors q for the new set of temperature snapshots based on the set of temperature factors τ and the set of thermal loading coefficients F. Step 332 can include constructing the spatial displacement field u of the modeled substrate for the new set of temperature snapshots based on the set of displacement factors q and the displacement basis U.

Step 334 can include displaying, at a display device, a graphical representation illustrating the spatial displacement field u for the ensemble of layers of the modeled substrate.

### 4. Zonal Structural Reduced Order Modeling

The method described by Equations (1)-(3) can be applied to the entire set of substrates as discussed above or can be applied for each layer. In fact, a unique basis can be found that represents the displacements of all of the layers (regardless of material) for all temperature cases (regardless of top or bottom of the substrate and of the temperature applied). This suggests that this same basis could also be used for other dies thereby eliminating the need to run full finite element analysis solutions to construct the basis as in the global ROM approach. The identification of the coefficients of these zonal ROMs would also be facilitated by the small size of the associated finite element model. The Zonal modeling steps outlined herein consider each layer independently and thus requires an appropriate handling of the necessary continuity of the displacements across layers.

A processor implementing the Zonal ROM process can receive a set of model parameters expressive of a modeled substrate having a plurality of layers, a set of simulation parameters and a set of physical properties. The processor can then determine a common basis indicative of displacement for the plurality of layers of the modeled substrate for a plurality of temperature cases through a Proper Orthogonal Decomposition (POD) of ensemble of displacements of layers obtained in existing finite element analysis solutions. The processor can determine a set of displacement factors for each respective layer of the plurality of layers of the modeled substrate using the set of model parameters and the set of simulation parameters through application of the Zonal ROM methodology.

One important distinction between Zonal ROM and Global ROM is the need to satisfy at best the continuity of the displacements at the interface between every pair of consecutive layers. These displacements are expressed as:

u_{n,n+1}=Φ_{b}qₙ

when considering the interface between an n^{th} layer and an (n+1)^{th} layer as part of the n^{th} layer; or

u_{n,n+1}=Φₜqₙ₊₁

when considering the same interface as part of the (n+1)^{th} layer, where qₙ and qₙ₊₁ are displacement factors of zonal reduced order modeling for the n^{th} layer and the (n+1)^{th} layer, where Φ_{b} is a component of the common basis that relate to nodes at a bottom of the associated layer and where Φₜ is a component of the common basis that relate to nodes at a top of the associated layer and where w_{n,n+1} is indicative of displacement at the n^{th} layer and the (n+1)^{th} layer.

The system can select qₙ and qₙ₊₁ that minimize a difference between the displacement w_{n,n=1} at the n^{th} layer and the (n+1)^{th} layer. In some embodiments, the common basis is a single basis in which each mode of the common basis includes components along an x direction and a y direction and along a z direction. In one example, the single basis includes 10 POD eigenvectors. In other embodiments, wherein common basis is a split basis in which one or more modes of the common basis include a set of in-plane components along an x direction and a y direction and one or more modes of the common basis include a set of transverse components along a z direction. In one example, the set of in-plane components includes 3 eigenvectors of in-plane displacement and the set of transverse components includes 7 eigenvectors of transverse displacement. Continuity can be enforced through a penalty handling methodology.

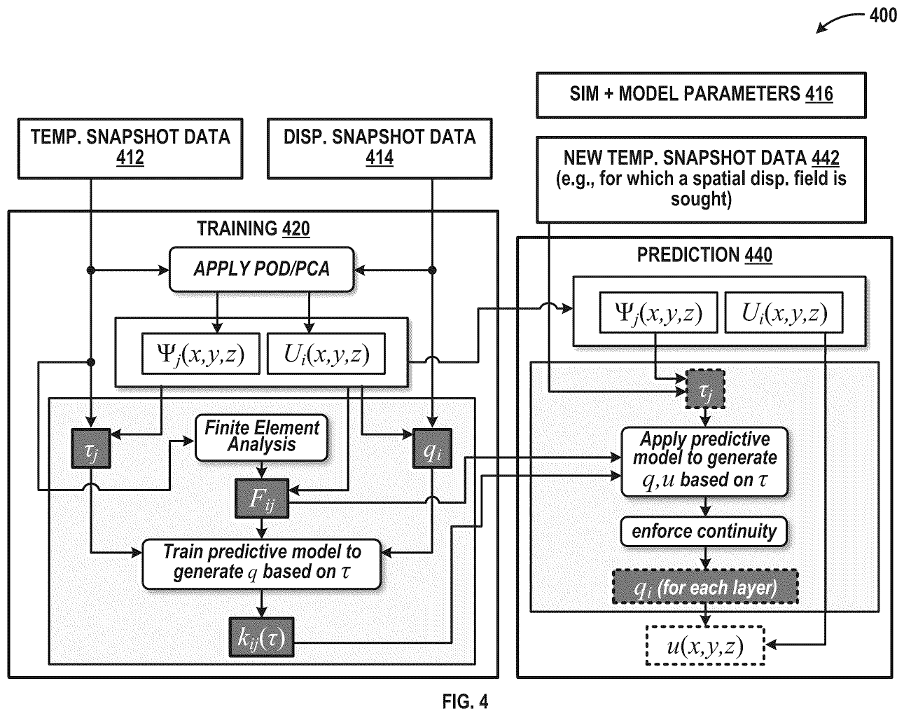

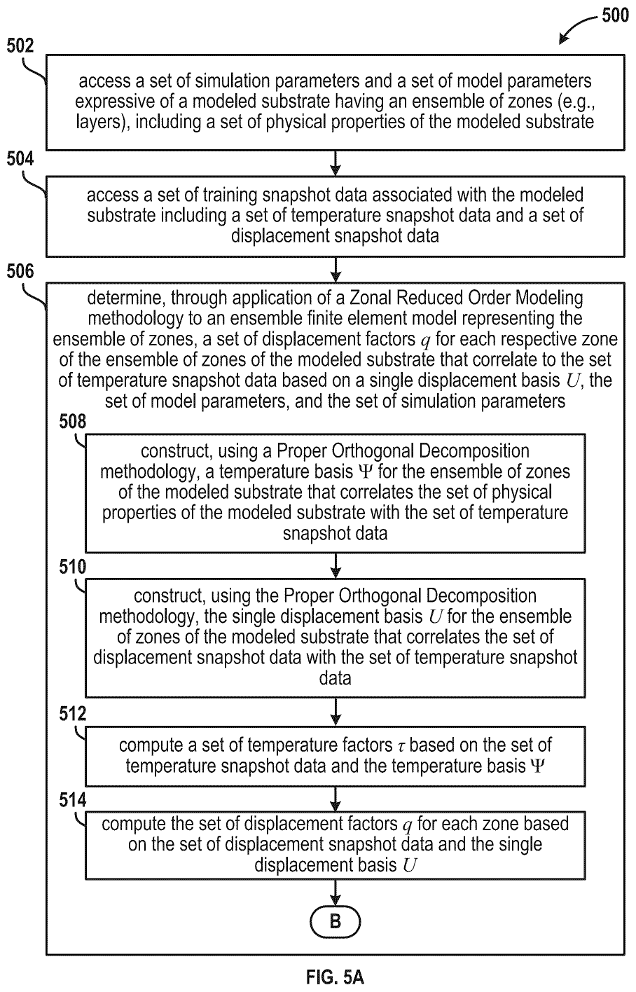

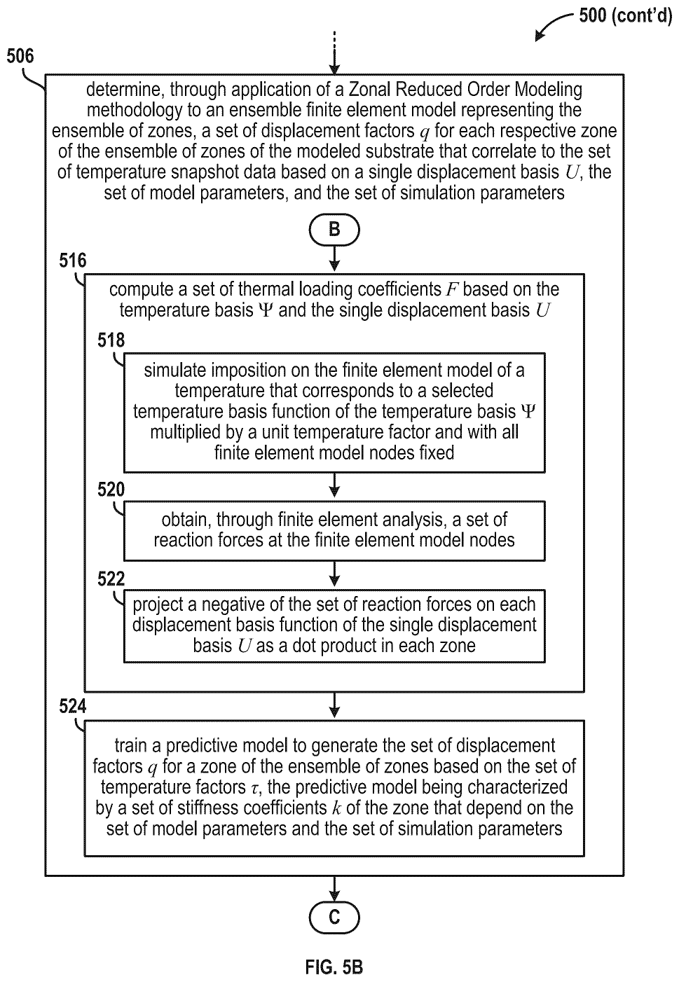

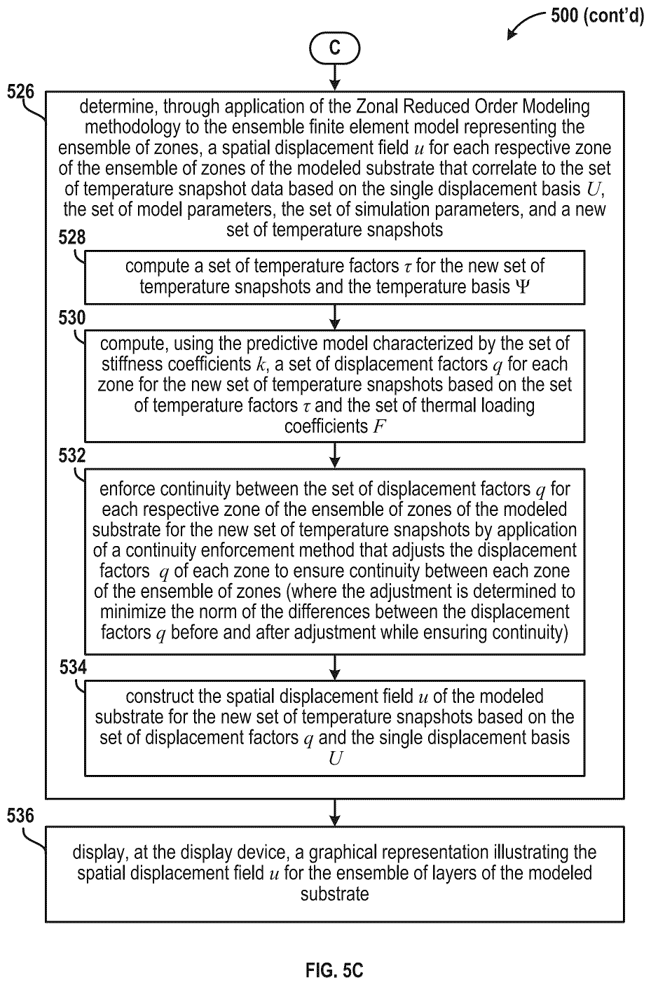

To determine a spatial displacement field u(x, y, z) for a modeled substrate having an ensemble of zones (more specifically, layers), a computing device (including a processor) implementing Zonal ROM 400 as conceptually illustrated in FIG. 4 and a second process 500 of FIGS. 5A-5C can first access a set of simulation parameters and a set of model parameters expressive of the modeled substrate, including a set of physical properties of the modeled substrate. For Zonal ROM, the basis functions can be broadly applied to all “zones” or “layers” of the modeled substrate, but other vectors and parameters can be found for individual zones. For some practical applications, this may be more computationally efficient than Global ROM because although Zonal ROM still requires determination of the basis functions for the ensemble of zones, concurrent calculation of displacements for individual zones may take significantly less time than Global ROM. The Zonal ROM can include a “training” block 420 and a “prediction” block 440 as shown in FIG. 4. Further, in FIG. 4, the dark boxes (corresponding to the set of temperature factors τⱼ, the thermal loading coefficients Fᵢⱼ, the set of displacement factors qᵢ, and the set of stiffness coefficients kᵢⱼ) are determined separately for each zone of the plurality of zones. Also, in FIG. 4, boxes that are in dotted lines (the set of temperature factors τⱼ, the set of displacement factors qᵢ, and the spatial displacement field u(x, y, z) within the “prediction” block 440) are quantities that can change when new data is being applied. In contrast, the thermal loading coefficients Fᵢⱼ and the set of stiffness coefficients kᵢⱼ can remain constant once training is performed within the “training” block 420.

Training data employed by the “training” block 420 can include “snapshots” of temperature distribution (“a set of temperature snapshot data 412”) throughout a modeled substrate, as well as “snapshots” of structural deformation (“a set of displacement snapshot data 414”) throughout the modeled substrate that correlate with temperature distribution. The set of displacement snapshot data 414 and the set of temperature snapshot data 412 for the modeled substrate can be obtained through existing finite element analysis solutions of the modeled substrate. Other information used by the “training” block 420 can include simulation parameters and model parameters (shown in FIG. 4 as “Sim+Model Parameters 416). Based on the set of temperature snapshot data 412, the set of displacement snapshot data 414, the computing device can construct, using a Proper Orthogonal Decomposition methodology, a temperature basis Ψ and a single displacement basis U that is valid for each zone of the ensemble of zones of the modeled substrate that correlates the set of displacement snapshot data 414 with the set of temperature snapshot data 412. By individually applying finite element analysis to individual zones (or more specifically, layers) of the modeled substrate in view of the temperature basis Ψ and the single displacement basis U, the computing device can find a set of temperature factors τ and a set of displacement factors q for the modeled substrate that correspond with one another. The set of displacement factors q can then be used to train a predictive model to generate sets of displacement factors q based on a given set of temperature factors τ. The illustration of FIG. 2B is similarly applicable for Zonal ROM. The set of displacement factors q are dependent upon the set of temperature factors τ expressive of a distribution, the set of model parameters, and the set of simulation parameters. Further, the set of displacement factors q can be determined from a set of stiffness coefficients (of a stiffness matrix k) and a set of thermal loading coefficients F both associated with each zone of the ensemble of zones of the modeled substrate.

Once the predictive model is trained, at the “prediction” block 440, the temperature basis Ψ and the single displacement basis U can be used to generate a new set of displacement factors q for each respective zone in view of more temperature cases, e.g., in view of a set of new temperature snapshot data 442. The new set of displacement factors q can be used along with the displacement basis U to generate the spatial displacement field u(x, y, z) for the entire modeled substrate.

FIGS. 5A-5C show the second process 500 that includes steps 502-524 performed with respect to the “training” block 420 and steps 526-536 are performed with respect to the “prediction” block 440.

Referring to FIG. 5A, step 502 of second process 500 includes accessing a set of simulation parameters and a set of model parameters expressive of a modeled substrate having an ensemble of zones (or, more specifically, layers), including a set of physical properties of the modeled substrate. Step 504 includes accessing a set of training snapshot data associated with the modeled substrate including a set of temperature snapshot data and a set of displacement snapshot data. Step 506 includes determining, through application of a Zonal Reduced Order Modeling methodology to an ensemble finite element model representing the ensemble of zones, a set of displacement factors q for each respective zone of the ensemble of zones of the modeled substrate that correlate to the set of temperature snapshot data based on a single displacement basis U, the set of model parameters, and the set of simulation parameters.

Step 506 can include several sub-steps 508-524. Steps 508 and 510 pertain to the basis functions for temperature and displacement that can be applied across the entire substrate, similar to steps 308 and 310 of the first process 300 for Global ROM. Step 508 includes constructing, using a Proper Orthogonal Decomposition methodology, a temperature basis Ψ for the ensemble of zones of the modeled substrate that correlates the set of physical properties of the modeled substrate with the set of temperature snapshot data. Step 510 includes constructing, using the Proper Orthogonal Decomposition methodology, the single displacement basis U for the ensemble of zones of the modeled substrate that correlates the set of displacement snapshot data with the set of temperature snapshot data.

Steps 512, 514 and 516 differ from analogous steps 312, 314, and 316 of the first process because, rather than being applied across the entire modeled substrate as in Global ROM, steps 514 and 516 are applied individually to each respective zone of the ensemble of zones of the modeled substrate. Steps 512, 514 and 516 pertain to determination of temperature factors that correspond to the temperature basis Ψ, displacement factors that correspond to the displacement basis U, and a set of thermal loading coefficients F that correlate the temperature basis Ψ with the single displacement basis U. Step 512 includes computing a set of temperature factors τ based on the set of temperature snapshot data and the temperature basis Ψ (using projection, e.g., a dot product or a least-squares process). Step 514 includes computing, for each respective zone of the ensemble of zones, a set of displacement factors q based on the set of displacement snapshot data and the single displacement basis U (using projection, e.g., a dot product or a least-squares process). FIG. 5A ends at (B), and continues the second process 500 at (B) of FIG. 5B starting with step 516. Referring to FIG. 5B, step 516 includes computing, for each respective zone of the ensemble of zones, a set of thermal loading coefficients F based on the temperature basis Ψ and the single displacement basis U. Note that the set of temperature factors τ calculated in step 512 remain global quantities even though the set of displacement factors q are zonal quantities.

Step 516 can include several sub-steps 518-522. Step 518 includes simulating imposition on the finite element model of a temperature that corresponds to a selected temperature basis function of the temperature basis Ψ multiplied by a unit temperature factor and with all finite element model nodes fixed. Step 520 includes obtaining, through finite element analysis, a set of reaction forces at the finite element model nodes. Step 522 includes projecting a negative of the set of reaction forces on each displacement basis function of the single displacement basis U as a dot product in each zone. Step 516 results in one set of training thermal loading coefficients F for each pair of temperature basis function and displacement basis function (e.g., an ij-th set of training thermal loading coefficients Fᵢⱼ for a j-th temperature basis function Ψⱼ paired with an i-th displacement basis function Uᵢ, and one such set of thermal loading coefficients for each zone).

Following step 516 (including sub-steps 518-522), step 524 includes training a predictive model to generate the set of displacement factors q based on the set of temperature factors τ. With additional reference to FIG. 2B, the predictive model can be characterized at least in part by a set of stiffness coefficients k that depend on the set of model parameters and the set of simulation parameters. Each displacement factor qᵢ can be written as linear combination of the training thermal loading coefficients Fᵢⱼ corresponding to that displacement basis function Uᵢ, scaled by the corresponding training vector of temperature factors τⱼ. The coefficients of the linear combination form the inverse of a stiffness matrix k for the zone, the elements of which are polynomials of the set of temperature factors τ. The act of training the predictive model determines the coefficients of the polynomials in the temperature factors τ of the elements of the stiffness matrix k that lead to the smallest error between the displacement factors q obtained by the predictive model and those found in step 514. FIG. 5B ends with (C) after conclusion of step 524 (of step 506), and continues at (C) of FIG. 5C.

Starting at (C) of FIG. 5C, the basis functions and the trained predictive model can then be applied to a new set of temperature snapshots (e.g., new set of temperature snapshot data 242) for construction of a spatial displacement field u of the modeled substrate for the new set of temperature snapshots. Steps 526-536 of second process 500 pertain to the “prediction” block 440 of FIG. 4. Note that unlike the Global ROM, for Zonal ROM steps 526-530 are performed for each respective zone of the ensemble of zones of the modeled substrate. Zonal ROM also requires an additional enforcement continuity method (outlined in step 532) in order to combine predictions into a cohesive spatial displacement field u for the entire modeled substrate.

Step 526 includes determining, through application of the Zonal Reduced Order Modeling methodology to the ensemble finite element model representing the ensemble of zones, a spatial displacement field u for each respective zone of the ensemble of zones of the modeled substrate that correlate to the set of temperature snapshot data based on the single displacement basis U, the set of model parameters, the set of simulation parameters, and a new set of temperature snapshots. Step 526 can include several sub-steps 528-534. Step 528 can include computing a set of temperature factors τ for the new set of temperature snapshots and the temperature basis function Ψ (using projection, e.g., a dot product or a least-squares process). Step 530 can include computing, using the predictive model characterized by the set of stiffness coefficients k, a set of displacement factors q for each zone and for the new set of temperature snapshots based on the set of temperature factors τ and the set of thermal loading coefficients F.

Step 532 includes enforcing continuity between the set of displacement factors q for each respective zone of the ensemble of zones of the modeled substrate for the new set of temperature snapshots. Step 532 is achieved by application of a continuity enforcement method that adjusts the displacement factors q of each zone as obtained from the trained predictive model to ensure continuity between each zone of the ensemble of zones (where the adjustment is determined to minimize the norm of the differences between the displacement factors q before and after adjustment while ensuring continuity). This is different from Global ROM, which is inherently continuous. For Zonal ROM, it is necessary to enforce continuity between predictions for each zone as the predictions are made individually for each zone.

Step 534 can include constructing the spatial displacement field u of the modeled substrate for the new set of temperature snapshots based on the set of displacement factors q for each zone and the single displacement basis U. The spatial displacement field u is an assembly of the displacements of each zone, and may be constructed by combining a plurality of spatial displacement fields pertaining to each individual zone into a total spatial displacement field u for the modeled substrate. Step 536 can include displaying, at a display device, a graphical representation illustrating the spatial displacement field u for the ensemble of layers of the modeled substrate.

### 5. Layer-by-Layer Computations Method

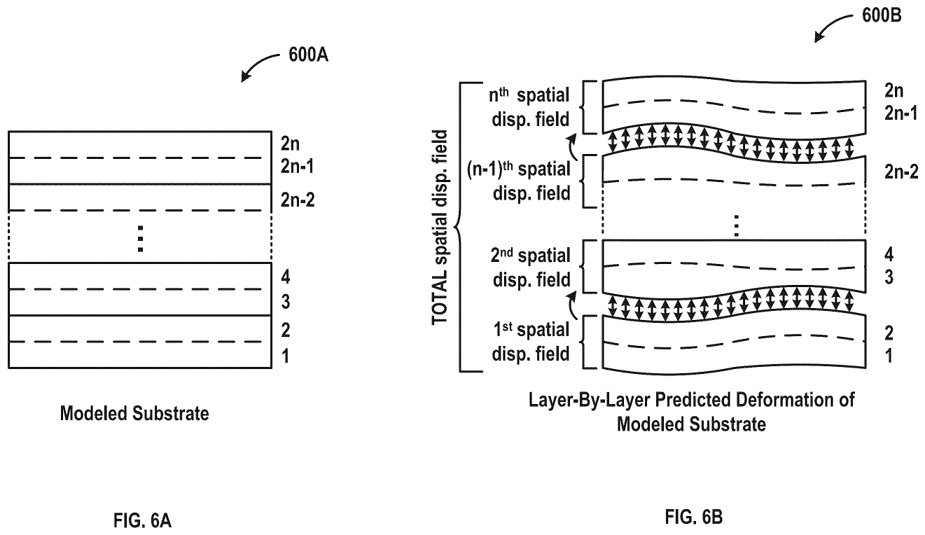

The third method described here is the layer-by-layer approach. According to this method, the displacements are determined by groups of 2-layers starting at the bottom toward the top. The displacement at the lowest of the two layers is the displacement obtained for the top of the previous set of 2 layers. This method effectively views the substrate as a weakly coupled chain of substructures (each being 2 consecutive layers). In some aspects, the third method can be beneficial by breaking down a large system into smaller, computationally manageable pieces (e.g., a multi-dimensional 500×500 problem to solving 25 2×2 problem). In other words, the layer-by-layer approach can allow solving a 24 layer problem using 12 iterations of 2×2 problems. FIGS. 6A and 6B provide an illustration of this concept.

In FIG. 6A, a modeled substrate 600A is shown having 2n layers, with each layer being paired with another adjacent layer that shares an interface (e.g., layer 1 is paired with layer 2, layer 3 is paired with layer 4, layer (2n−1) is paired with layer 2n, etc.). As shown, while layer 1 is paired with layer 2 and so forth, layers 2 and 3 share an interface. When determining displacements for the modeled substrate 600A using the layer-by-layer approach, finite element modeling can be used to jointly predict or otherwise determine a first spatial displacement field for layers 1 and 2 (where layer 1 is on the bottom, and layer 2 is above layer 1 and shares an interface). Finite element modeling can then be used to jointly predict or otherwise determine a second spatial displacement field for layers 3 and 4, working upwards. To ensure continuity, the bottom of the second spatial displacement field associated with the bottom of layer 3 should match the top of the first spatial displacement field associated with the top of layer 2, and so forth.

FIG. 6B shows deformation of a modeled substrate 600B in which a first spatial displacement field is determined with respect to layers 1 and 2, a second spatial displacement field is subsequently determined with respect to layers 3 and 4 (ensuring continuity at the interface between layers 2 and 3), and so forth. FIG. 6B shows an n^{th} spatial displacement field for a (2n−1)^{th} layer and a (2n)^{th} layer of the ensemble of layers of the modeled substrate 600B, the (2n−1)^{th} layer being below the (2n)^{th} layer and sharing an interface with a top of a (2n−2)^{th} layer, the n^{th} spatial displacement field at a bottom of the (2n−1)^{th} layer being equal to an (n−1)^{th} spatial displacement field at the top of the (2n−2)^{th} layer. Spatial displacement fields 1 through n can be combined into a total spatial displacement field (akin to u(x, y, z) discussed for the Global ROM and Zonal ROM methods outlined above).

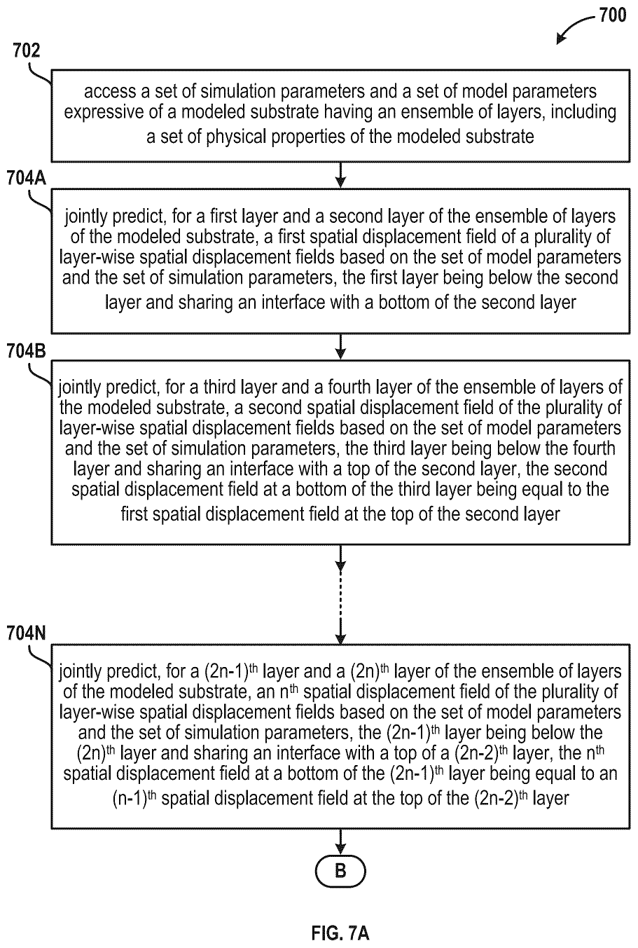

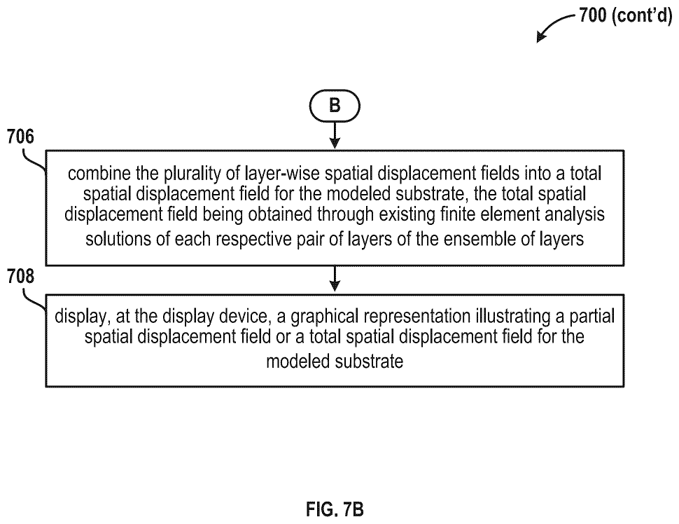

FIGS. 7A and 7B outline a third process 700 that may be implemented by a computing system for predicting a total spatial displacement field according to the Layer-By-Layer approach discussed above with respect to FIGS. 6A and 6B.

With reference to FIG. 7A, step 702 of third process 700 includes accessing a set of simulation parameters and a set of model parameters expressive of a modeled substrate having an ensemble of layers, including a set of physical properties of the modeled substrate. Steps 704A-704N pertain to sequential prediction/determination of spatial displacement fields 1 through n as discussed above. Step 704A includes jointly predicting, for a first layer and a second layer of the ensemble of layers of the modeled substrate, a first spatial displacement field of a plurality of layer-wise spatial displacement fields based on the set of model parameters and the set of simulation parameters, the first layer being below the second layer and sharing an interface with a bottom of the second layer. Step 704B includes jointly predicting, for a third layer and a fourth layer of the ensemble of layers of the modeled substrate, a second spatial displacement field of the plurality of layer-wise spatial displacement fields based on the set of model parameters and the set of simulation parameters, the third layer being below the fourth layer and sharing an interface with a top of the second layer, the second spatial displacement field at a bottom of the third layer being equal to the first spatial displacement field at the top of the second layer. This can continue in this pattern for the remaining layers. For a final pair of layers, step 704N can include jointly predicting, for a (2n−1)^{th} layer and a (2n)^{th} layer of the ensemble of layers of the modeled substrate, an n^{th} spatial displacement field of the plurality of layer-wise spatial displacement fields based on the set of model parameters and the set of simulation parameters, the (2n−1)^{th} layer being below the (2n)^{th} layer and sharing an interface with a top of a (2n−2)^{th} layer, the n^{th} spatial displacement field at a bottom of the (2n−1)^{th} layer being equal to an (n−1)^{th} spatial displacement field at the top of the (2n−2)^{th} layer.

Referring to FIG. 7B, step 706 includes combining the plurality of layer-wise spatial displacement fields into a total spatial displacement field for the modeled substrate, the total spatial displacement field being obtained through existing finite element analysis solutions of each respective pair of layers of the ensemble of layers. Step 708 includes displaying, at a display device, a graphical representation illustrating a partial spatial displacement field or a total spatial displacement field for the modeled substrate.

### 6. Computer-Implemented System

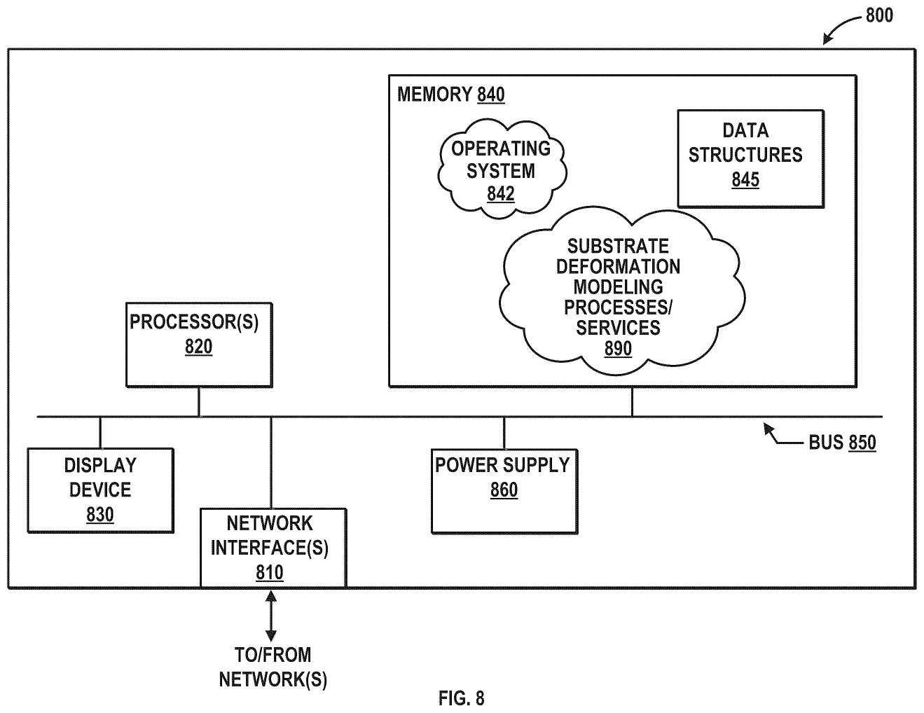

FIG. 8 is a schematic block diagram of an example device 800 that may be used with one or more embodiments described herein, e.g., as a computing device implementing aspects of the Global ROM (first process 300 of FIGS. 3A-3C), the Zonal ROM (second process 500 of FIGS. 5A-5C), and/or Layer-by-Layer (third process 700 of FIGS. 7A and 7B).

Device 800 comprises one or more network interfaces 810 (e.g., wired, wireless, PLC, etc.), at least one processor 820, and a memory 840 interconnected by a system bus 850, as well as a power supply 860 (e.g., battery, plug-in, etc.). Device 800 can further include a display device 830 operable for displaying a graphical representation illustrating the spatial displacement field u for the ensemble of layers of the modeled substrate.

Network interface(s) 810 include the mechanical, electrical, and signaling circuitry for communicating data over the communication links coupled to a communication network. Network interfaces 810 are configured to transmit and/or receive data using a variety of different communication protocols. As illustrated, the box representing network interfaces 810 is shown for simplicity, and it is appreciated that such interfaces may represent different types of network connections such as wireless and wired (physical) connections. Network interfaces 810 are shown separately from power supply 860, however it is appreciated that the interfaces that support PLC protocols may communicate through power supply 860 and/or may be an integral component coupled to power supply 860.

Memory 840 includes a plurality of storage locations that are addressable by processor 820 and network interfaces 810 for storing software programs and data structures associated with the embodiments described herein. In some embodiments, device 800 may have limited memory or no memory (e.g., no memory for storage other than for programs/processes operating on the device and associated caches). Memory 840 can include instructions executable by the processor 820 that, when executed by the processor 820, cause the processor 820 to implement aspects of the systems and one or more of the processes 300, 500 or 700 outlined herein.

Processor 820 comprises hardware elements or logic adapted to execute the software programs (e.g., instructions) and manipulate data structures 845. An operating system 842, portions of which are typically resident in memory 840 and executed by the processor, functionally organizes device 800 by, inter alia, invoking operations in support of software processes and/or services executing on the device. These software processes and/or services may include Substrate Deformation Modeling processes/services 890, which can include aspects of one or more of the processes 300, 500 or 700 and/or implementations of various modules described herein. Note that while Substrate Deformation Modeling processes/services 890 is illustrated in centralized memory 840, alternative embodiments provide for the process to be operated within the network interfaces 810, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the Substrate Deformation Modeling processes/services 890 is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a processor in communication with a memory, the memory including instructions executable by the processor to:
access a set of simulation parameters and a set of model parameters expressive of a modeled substrate having an ensemble of zones, including a set of physical properties of the modeled substrate;
construct, using a Proper Orthogonal Decomposition methodology, a displacement basis for the ensemble of zones of the modeled substrate that correlates a set of displacement snapshot data with a set of temperature snapshot data;
determine, through application of a global reduced order modeling methodology to an ensemble finite element model representing the ensemble of zones, a set of displacement factors for the ensemble of zones of the modeled substrate that correlate to the set of temperature snapshot data based on the displacement basis, the set of model parameters, and the set of simulation parameters; and
determine a spatial displacement field of the modeled substrate for a plurality of temperature cases based on the set of displacement factors and the displacement basis.

2. The system of claim 1, the memory further including instructions executable by the processor to:
construct, using a Proper Orthogonal Decomposition methodology, a temperature basis for the ensemble of zones of the modeled substrate that correlates the set of physical properties of the modeled substrate with the set of temperature snapshot data.

3. The system of claim 1, further comprising:
a display device in communication with the processor, the memory further including instructions executable by the processor to:
display, at the display device, a graphical representation illustrating the spatial displacement field for the ensemble of layers of the modeled substrate.

4. The system of claim 1, the set of displacement snapshot data and the set of temperature snapshot data for the modeled substrate being obtained through existing finite element analysis solutions of a substrate.

5. The system of claim 1, the set of displacement factors being dependent upon a set of temperature factors expressive of a distribution, the set of model parameters, and the set of simulation parameters, and the set of displacement factors being determined from a set of stiffness coefficients and a set of thermal loading coefficients.

6. The system of claim 5, the memory further including instructions executable by the processor to:
access the set of displacement factors and the set of temperature snapshot data; and
train a predictive model to generate the set of displacement factors based on the set of temperature factors using the set of stiffness coefficients that depend on the set of model parameters and the set of simulation parameters.

7. The system of claim 1, wherein the displacement basis is a split basis in which one or more modes of a plurality of modes of the displacement basis include a set of in-plane components along an x-direction and a y-direction of the modeled substrate, and one or more modes of the plurality of modes of the displacement basis include a set of transverse components along a z-direction of the modeled substrate.

8. A system, comprising:
a processor in communication with a memory, the memory including instructions executable by the processor to:
access a set of simulation parameters and a set of model parameters expressive of a modeled substrate having an ensemble of zones, including a set of physical properties of the modeled substrate;
construct, using a Proper Orthogonal Decomposition methodology, a single displacement basis that is valid for each zone of the ensemble of zones of the modeled substrate that correlates a set of displacement snapshot data with a set of temperature snapshot data;
determine, through application of a Zonal Reduced Order Modeling methodology to an ensemble finite element model representing the ensemble of zones, a set of displacement factors for each respective zone of the ensemble of zones of the modeled substrate that correlate to the set of temperature snapshot data based on the single displacement basis, the set of model parameters, and the set of simulation parameters; and
determine a spatial displacement field of the modeled substrate for a plurality of temperature cases based on the set of displacement factors and the single displacement basis.

9. The system of claim 8, the memory further including instructions executable by the processor to:
construct, using a Proper Orthogonal Decomposition methodology, a temperature basis for the ensemble of zones of the modeled substrate that correlates the set of physical properties of the modeled substrate with the set of temperature snapshot data.

10. The system of claim 8, further comprising:
a display device in communication with the processor, the memory further including instructions executable by the processor to:
display, at the display device, a graphical representation illustrating the spatial displacement field for the modeled substrate.

11. The system of claim 8, the set of displacement factors being dependent upon a set of temperature factors expressive of a temperature distribution, the set of model parameters, and the set of simulation parameters, and the set of displacement factors being determined from a set of stiffness coefficients and a set of thermal loading coefficients associated with each zone of the ensemble of zones of the modeled substrate.

12. The system of claim 11, the memory further including instructions executable by the processor to:
access the set of displacement factors and the set of temperature factors; and
train a predictive model to generate the set of displacement factors for a zone of the ensemble of zones based on the set of temperature factors of the zone using the set of stiffness coefficients of the zone that depend on the set of model parameters and the set of simulation parameters.

13. The system of claim 8, the memory further including instructions executable by the processor to:
enforce continuity between the set of displacement factors for each respective zone of the ensemble of zones of the modeled substrate.

14. The system of claim 13, the memory further including instructions executable by the processor to:
determine the smallest adjustments to the set of displacement factors obtained from the trained predictive model that lead to continuity between spatial displacements at shared interfaces of each respective zone of the modeled substrate.

15. The system of claim 8, the set of displacement snapshot data and the set of temperature snapshot data for the modeled substrate being obtained through existing finite element analysis solutions of a substrate.

16. The system of claim 8, wherein the single displacement basis is a split basis in which one or more modes of a plurality of modes of the single displacement basis include a set of in-plane components along an x-direction and a y-direction of the modeled substrate, and one or more modes of the plurality of modes of the single displacement basis include a set of transverse components along a z-direction of the modeled substrate.

17. A system, comprising:
a processor in communication with a memory, the memory including instructions executable by the processor to:
access a set of simulation parameters and a set of model parameters expressive of a modeled substrate having an ensemble of layers, including a set of physical properties of the modeled substrate;
jointly predict, for a first layer and a second layer of the ensemble of layers of the modeled substrate, a first spatial displacement field of a plurality of layer-wise spatial displacement fields based on the set of model parameters and the set of simulation parameters, the first layer being below the second layer and sharing an interface with a bottom of the second layer; and
jointly predict, for a third layer and a fourth layer of the ensemble of layers of the modeled substrate, a second spatial displacement field of the plurality of layer-wise spatial displacement fields based on the set of model parameters and the set of simulation parameters, the third layer being below the fourth layer and sharing an interface with a top of the second layer, the second spatial displacement field at a bottom of the third layer being equal to the first spatial displacement field at the top of the second layer.

18. The system of claim 17, the memory further including instructions executable by the processor to:
jointly predict, for a (2n−1)^{th} layer and a (2n)^{th} layer of the ensemble of layers of the modeled substrate, an n^{th} spatial displacement field of the plurality of layer-wise spatial displacement fields based on the set of model parameters and the set of simulation parameters, the (2n−1)^{th} layer being below the (2n)^{th} layer and sharing an interface with a top of a (2n−2)^{th} layer, the n^{th} spatial displacement field at a bottom of the (2n−1)^{th} layer being equal to an (n−1)^{th} spatial displacement field at the top of the (2n−2)^{th} layer.

19. The system of claim 17, the memory further including instructions executable by the processor to:
combine the plurality of layer-wise spatial displacement fields into a total spatial displacement field for the modeled substrate, the total spatial displacement field being obtained through existing finite element analysis solutions of each respective pair of layers of the ensemble of layers.

20. The system of claim 17, further comprising:
a display device in communication with the processor, the memory further including instructions executable by the processor to:
display, at the display device, a graphical representation illustrating a partial spatial displacement field or a total spatial displacement field for the modeled substrate.
